(19) United States
(12) Patent Application Publication
(10) Pub. No.: US 2013/0243887 A1
(43) Pub. Date: Sep. 19, 2013

(54) ZEOLITES HAVING A NEURO-PROTECTIVE EFFECT

(71) Applicants: Maurizio Memo, Brescia (IT); Stefano Manna, Roma (IT)

(72) Inventors: Maurizio Memo, Brescia (IT); Stefano Manna, Roma (IT)

(73) Assignees: Jakob Hraschan, DROBOLACH (AT); ECOBASE GMBH, LIESERBRUECKE (AT); Martina Ricco' Dullnig, LIESERBRUECKE (AT)

(21) Appl. No.: 13/813,758

(22) PCT Filed: Aug. 3, 2011
(86) PCT No.: PCT/IB11/53461

§ 371 Date: Feb. 1, 2013

(30) Foreign Application Priority Data
Aug. 3, 2010 (IT) RM2010A000435

(51) Int. Cl. A61K 33/06

(52) U.S. Cl. CPC A61K 33/06
USPC 424/684; 423/700

(57) ABSTRACT

The present invention relates to the preparation of zeolites obtained with a method comprising a stage of heating to temperature preferably of over 400° C. and at least one stage of micronisation. Such zeolites are to be used in the pharmaceutical sector, in particular to combat the action of ROS at a mitochondrial level with a neuroprotective effect.

### FIELD OF THE INVENTION

The present invention relates to zeolites having a neuro-protective effect. In particular the invention relates to an activated clinoptilolite zeolite having elevated anti-oxidant activity in the mitochondria and a neuro-protective effect, therefore able to be of benefit in those neuropathologies where the production of ROS plays a significant role, such as Alzheimer's or Parkinson's disease.

### PRIOR ART

The reactive oxygen species (ROS), such as H₂O₂ and the radicals OH. and O₂., perform important physiological functions (e.g. macrophages) but can also cause vast cellular damage, so-called oxidative stress, an imbalance between the production of ROS and the cell's anti-oxidant defences which may affect lipids, proteins, carbohydrates and DNA. This phenomenon has been associated with a series of human illnesses such as cardiovascular disease, diabetes, cancer and neuro-degenerative disease.

Zeolites include about 150 different types of compound, of natural or synthetic origin with varying chemical-physical structures. In chemical terms zeolites are the aluminosilicates of sodium, potassium, calcium or magnesium.

Zeolites are crystalline substances the structure of which is characterised by a lattice consisting of tetrahedrons bound to each other by oxygen bridges. The crystalline lattice contains empty spaces in the form of cages and channels, the latter usually occupied by molecules of H₂O and other cations, which may be exchanged. The channels are sufficiently wide to permit the transit of host molecules. The water rich phases may dehydrate: dehydration usually occurs at temperature below 400° C., and is widely reversible.

The clinoptilolite zeolite is of volcanic origin, highly stable compared to other types of zeolite and is particularly suitable for applications in the pharmaceutical field. It has a crystalline structure consisting of two tetrahedrons of SiO₄ and AlO₄ bound by oxygen bridges which generate free spaces and channels in which even relatively large cations and molecules can be received. The negative charges of the aluminate and silicate units are neutralised by the presence of cations such as protons, calcium, magnesium, sodium, potassium. These ions can be replaced by other cations, such as heavy metals or ammonium ion. This possibility is defined as the cation exchange capacity and is one of the main characteristics of the clinoptilolite.

Zeolites are used for a variety of technical and pharmaceutical applications related to their chemical-physical characteristics such as the cation exchange capacity, adsorption, molecular sieve and lastly as a support for catalysts utilisable in organic reactions.

A pharmaceutical application of zeolite clinoptilolite with dimensions of 0.5 μm was developed by T. Lelas in EP 1316530, achieved by means of a special micronisation process. The risks deriving from absorption of such small particles in the gastro-intestinal tract were recently pointed out in US 2009/0226492 by H. Danz et al. who created a micronised zeolite clinoptilolite for use in the pharmaceutical field suitable for absorbing heavy metals and having dimensions of 6 to 9 μm.

A modification of the micronisation system developed by A. Lelas in US 2009/133413 led to a zeolite clinoptilolite being obtained with high mesoporosity and even smaller dimensions of the particles between 100 nm and 2 μm with Si/Al ratios indicative of a zeolite rich in silicates. Such method aims to increase the ability of the particles to bind toxins, metals, bacteria and viruses which enter the skin and mucous through the gastro-intestinal tract in animal and human organisms so as to detoxify them.

According to the inventors, particles of such small dimensions increase the risk of their absorption in the gastro-intestinal tract with possible damage to cellular functionality and the additional risk of interaction between the particles which, as reported by A. Lelas, are suitable for binding molecules of greater dimensions as well as very large structures such as bacteria and viruses, with the consequent risk of stably binding proteins, essential oligo-elements and any medications associated with the zeolite, with inevitable risks to health due to the fact that the organism is not only deprived of potentially harmful molecules but also of those compounds essential for it to function.

Moreover, the purification of zeolite performed by Lelas in WO 2009/133413 in an acid environment entails a complication the benefit of which is doubtful in that the impurities generally present in natural zeolite are extremely low and are not even transferred in the acid gastric environment. Lastly, bearing in mind the scale of the cation exchange capacity, some doubts arise as to the possibility of a rapid exchange of the ammonium or calcium salts with other cations to obtain salts of a single chemical species, with consequent limitations of their application compared to natural zeolite containing more easily exchangeable cations. In fact the stability of the electrostatic interaction between the cations and the negative charges of the zeolite could slow down and/or limit the cation exchange capacity, as shown by the exemplificative test of C-MC (calcium salt of zeolite) with mercury, in which very low concentrations (60 ppb) were used which are not totally absorbed by 1 g of zeolite at pH 8.1. Conversely it was verified that the clinoptilolite zeolite which the present invention relates to is suitable for adsorbing 6 mg per 1 g of zeolite at pH 7, a quantity about 100 times greater.

To the knowledge of the inventors applications of zeolites to combat the action of ROS, in particular at a mitochondrial level and with a neuro-protective effect have not been studied.

### SUMMARY OF THE INVENTION

A method has now been found which the present invention relates to for obtaining a zeolite particularly efficacious in the medical field, especially to combat free radicals (ROS) which does not have the drawbacks of the known products.

The invention relates in particular to the zeolite clinoptilolite.

The zeolite of the invention is a dehydrated, micronised and activated zeolite characterised by a bimodal distribution of particles, as described herein, suitable for performing in particular a neuro-protective function at a mitochondrial level by means of a mechanism of oxidative stress reduction, being suitable for binding toxins and free radicals as well as heavy metal metals and the ammonium ion.

The method for obtaining the zeolite of the invention is characterised by a stage of heating the zeolite to a temperature of preferably over 400° C. and by subsequent micronisation and activation in one or more stages so as to obtain particles in the range of 1-60 μm and negative potential ζ of a corresponding value to that indicated in the description.

Further purposes will be evident from the detailed description of the invention which follows.

### BRIEF DESCRIPTION OF THE DRAWINGS

FIG. 1 is an NMR graph of the ZCD containing the global trends of the three samples A, B and C assayed;

FIG. 2 is an NMR graph of the non-activated ZCD, sample A;

FIG. 3 is an NMR graph of the ZCD activated once, sample B;

FIG. 4 is an NMR graph of the ZCD activated twice, sample C;

FIG. 5 shows the ROS (Reactive Oxygen Species) production in the mitochondria of SHSY-SY cells pre-treated with zeolite activated once, sample B;

FIG. 6 shows the ROS (Reactive Oxygen Species) production in the mitochondria of SHSY-SY cells pre-treated with zeolite activated twice, sample C;

FIG. 7 show the neuro-protective effect of different types of zeolite in SHSY-SY neuronal cells after exposure to H₂O₂. The cells were treated with different concentrations of Zeolite 24 hours before exposure to H₂O₂.

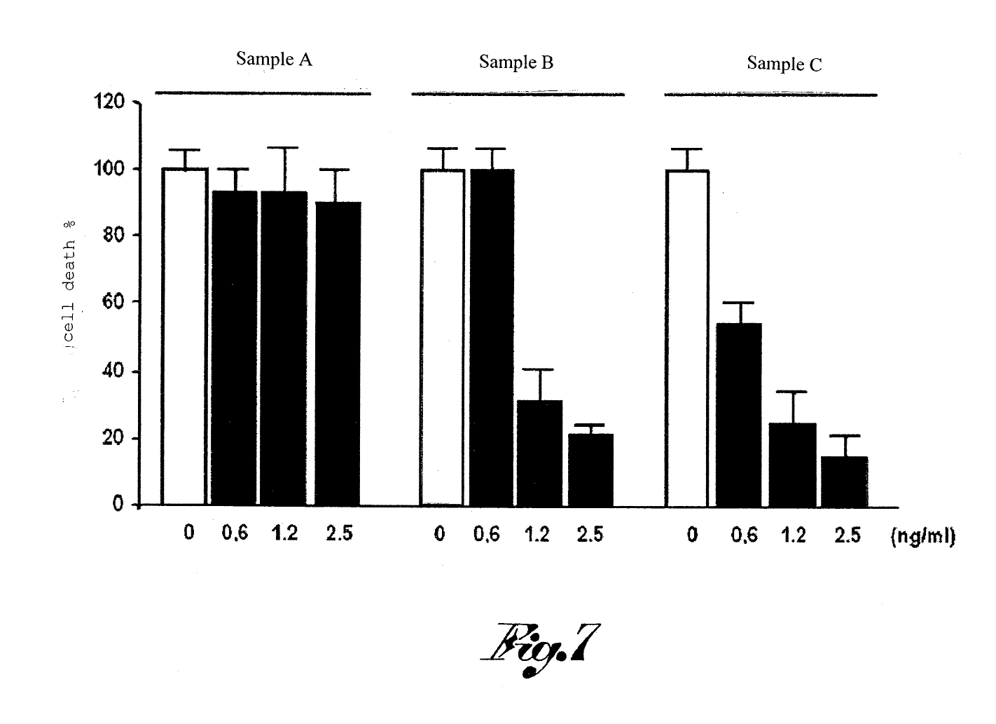

FIG. 8 shows the effect of dehydration on the neuro-protection induced by Zeolite activated once only, sample A and corresponding hydrate, in SHSY-SY neuronal cells after exposure to H₂O₂. The cells were treated with different concentrations of Zeolite 24 hours before exposure to H₂O₂.

### DETAILED DESCRIPTION OF THE INVENTION

According to the present invention, the term activation is understood to mean a structural variation deriving from a modification of the connectivity between the atoms of silicon and the atoms of aluminium in the lattice with a corresponding increase in the negative charges present on the particles, determining as a result of the rupture of the bond between the atom of aluminium and the bridging atom of oxygen with the silicon, an increase in the negative value of the potential and of the ion exchange capacity. Consequently, activated zeolite is understood to mean zeolite structurally modified as described above, deriving from the combined process of heating and micronisation according to the present invention. Micronisation may be conducted in one or more stages on previously ground material, in anhydrous conditions, to achieve 70%, preferably 80%, more preferably 85% and even more preferably 90% ratios of Si/Al of the zeolite in the range of 1:1-1:3.

Consequently, as mentioned above, the aforesaid ratios are not to be understood as “molar” Si/Al ratios in the lattice, because the chemical composition remains substantially unaltered before and after the activation phase, but as “connectivity” ratios; in fact, the heterolytic rupture of the oxygen-aluminium bonds in the lattice causes on the one hand an increase in charge density of the zeolite particles, on the other a reduction of the co-ordination degree between the atoms of silicon and the surrounding aluminium atoms.

The process of treating the zeolite is characterised by a heating stage to be conducted before micronisation at a temperature of preferably over 400° C., more preferably of 400-500° C., even more preferably 420-450° C., advantageously for about 15-30 minutes.

Preferably, the heating is conducted in a rotating drum. It is however possible to adopt temperatures of less than 400° C. for this stage on condition that treatment times are prolonged. During this heating phase there is a dehydration of the particles of zeolite which must be kept in anhydrous conditions for the subsequent micronisation treatment.

After the thermal treatment the zeolite is subjected to one or more stages of micronisation, typically using the process described in EP1316530, to obtain particles having dimensions in the range of 1-60 μm.

Advantageously, the micronisation treatment is conducted on a zeolite previously ground to an average dimension of 80-120 μm. Micronisation produces a modification of the size of the particles the characteristic of which is given by a shift in size to significantly smaller dimensions of 50-60 μm accompanied by an increase in particles of 8-35 μm.

Subsequent to the process according to the invention, that is to say after the combination of dehydration and micronisation in anhydrous conditions, there is a modification of the structure of the silicoaluminates, in particular of the Si/Al ratio with an increase in the structures characterised by values 1:2 and 1:1 and a reduction of those having values of 1:3 and 1:4, as explained below. Such modification entails a corresponding increase of the negative charges present on the particles which situate themselves on the atoms of oxygen liberated following rupture of some of the bonds which they formed as a bridge between the silicon and aluminium.

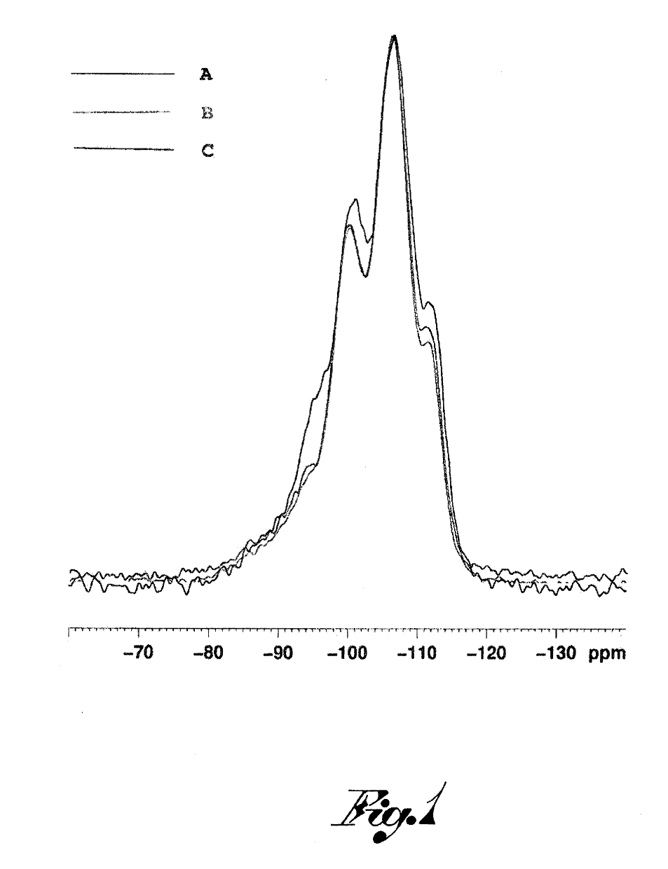

The extent of activation and structural modification with reduction of the Si/Al ratio is highlighted by the NMR analysis as shown in FIGS. 1-4.

From tests conducted on a sample examined by NMR, zeolite according to the invention is characterised by three main structures consisting of resonance bands at about −106.7 ppm T (1Al or 0Al) (46.81%) (ratio of peak areas of absorption under the NMR) in which each atom of silicon is bound by an oxygen bridge to an atom of aluminium with an Si/Al ratio of 1:1 and at about −100.82 T(2Al or 1Al) (24.68%) in which each atom of silicon is bound by an oxygen bridge to two atoms of aluminium with a Si/Al ratio of 1:2 with low percentages of resonance band at about −96.29 T(3Al or 2Al) (12.15%) consisting of silicon bound by an oxygen bridge to three atoms of aluminium with a Si/Al ratio of 1:3 small quantities of silicates and various silicoaluminates are also present in quantities of approximately less than 15% in weight.

The zeolite obtained using the method according to the invention is further characterised by a mesoporosity with pore diameter of about 30-40 Å, preferably about 35-38 Å, a surface of about 20-30 m²/g, preferably about 25-28 m²/g and lastly by a negative potential deriving from the superficial electrostatic charges present on the particles which determine an electric field responsible for the redistribution of the cations present in the space surrounding them equal or less than −26.6 mV, preferably in the range between −26.6 mV and −30 mV.

The negative values of potential increase with micronisation, demonstrating that an increase of the negative charges present on the particles occurs which coincides with the structural modifications highlighted by the NMR analysis. Such trend therefore demonstrates that the micronisation process does not just entail a reduction of the dimensions of the particles but also produces an activation of the same which increases their cation exchange capacity, further confirmed by the reduced transfer capacity in water of the ions present in their channels (Na⁺, K⁺, Ca²⁺, Mg²⁺) which are more strongly retained following the increase in the quantity of charge, especially in a neutral environment. Furthermore there is an increase in the neutralisation capacity of the free radicals following the greater availability of electrons suitable for forming permanent bonds with them.

The reduction of available charges deriving from the presence of water in the zeolite is highlighted by the lower value of the negative ζ potential of the hydrated zeolite activated only once.

The combination of characteristics described above allows the particles not to be absorbed at an intestinal level and to bind the free radicals more rapidly and permanently, explicating a neuro-protective function.

It has been experimentally demonstrated that the combination of heating and subsequent micronisation in anhydrous conditions causes a structural modification of the zeolite thus treated. Such structural modification has been studied as shown in the experimental part.

The zeolite clinoptilolite thus dehydrated (ZCD) and activated has been shown to be much more active than the hydrated equivalent in protecting from oxidative stress.

The zeolite obtained using the method according to the invention can be used in cosmetic, dietetic and pharmaceutical fields in various forms for use as a therapeutic agent in humans and in animals to counter the effects of the ROS at a mitochondrial level with a neuro-protective effect.

In particular the zeolite of the invention can be used to prevent and treat all those conditions and diseases which can be traced to the presence of ROS in the organism, therein including skin conditions such as damage from exposure to the sun, tumours or wounds and ulcers of the skin.

The aforesaid formulations may be: tablets, lozenges, capsules, syrups, suspensions, creams and unguents containing the zeolite of the invention as the active substance in quantities from 0.1 to 99.9% in weight in combination with one or more pharmaceutically acceptable excipients or carriers in quantities from 99.9 to 0.1% in weight.

The excipients are those known in the art and are, for example, chosen from the group comprising fillers, binders, lubricants, emollients, emulsifying agents, solvents, surfactants, moistening agents, preservatives, anti-oxidants, stabilisers, colorants, perfuming agents, pH controllers and other functional additives.

Other excipients to use to prepare the compositions depending on the specific administration route will be evident for anyone skilled in the art.

The formulations can be used to prepare pharmaceutical products, dietary products and food supplements or feeds or cosmetic products for humans and animals.

The compositions may further contain or be administered in association with vitamins, anti-inflammatory agents, anti-oxidants, hepatoprotective medicines, astringents for application in the gastrointestinal tract, anti-flogistics, chemotherapy and other food supplements known in themselves, therein including homoeopathy products and products of natural origin.

The formulations according to the present invention may be obtained directly by addition of the zeolite according to the invention to one or more excipients according to the standard methods known in the art [S. C. Gad (Ed.) Pharmaceutical Manufacturing Handbook: Production and Processes, Wiley (2008)].

The tablets may for example be obtained by means of compression, homogenisation and granulation operations.

The liquid formulations may be obtained by homogenisation of the zeolite with one or more excipients in water-based or hydro-alcoholic or oil-based liquid solutions, preferably with the addition of moistening agents in themselves known.

Lotions, emulsions and creams may be obtained by adding the zeolite according to the invention to the basic components of the aforesaid pharmaceutical forms such as fatty acids and emulsifying agents as well as any pharmaceutical or natural active substances. Moreover it may form part of the formulations of cosmetic products in their general applications for the body, eyes, face, legs, including combined with sun screens already in themselves known.

Unlike all the other anti-oxidants used in treatment, once the zeolite which the present invention relates to has been administered orally, it is not absorbed and acts in the intestine without interfering with the biological processes of the body. In addition it is not toxic and is characterised by the absence of any type of adverse reaction which is, on the contrary, present in all types of anti-oxidants, including vitamins which by acting in a systemic manner entail the risk of interference with various biological body processes. Furthermore, their metabolization may entail problems of interactions with medications, supplements, foods or substances taken contemporaneously to their administration, at a hepatic and renal level. The zeolite which the present invention relates to may be formulated and/or administrated with one or more adjuvants; in fact it does not interfere with any type of substance, including medicines. Such characteristic depends on its structure which does not allow interaction with lipophilic compounds having a high molecular weight such as medicines. In addition, all the active ingredients act systemically and are therefore rapidly absorbed in the intestine and once in circulation cannot interfere with the zeolite which remains in the intestine, not being absorbed.

The zeolite which the present invention relates to has a negative ζ potential value and a greater cation exchange capacity than the zeolites of the prior art; in addition it is free of any intestinal absorption problems, thereby preventing any risks of cellular toxicity.

Consequently, in the light of the previous description, it is evident how the present invention refers to a dehydrated and micronised clinoptilolite, characterised by a negative ζ potential equal to or less than −26.6 mV. Preferably, such potential is between −26.6 mV and −30 mV. Advantageously, moreover, the average dimensions of the aforementioned clinoptilolite are in the range 1-60 μm.

The present invention further refers to a method for preparing an activated clinoptilolite, preferably of the zeolite as per the previous paragraph, comprising, in sequence, the steps of:

i) providing a clinoptilolite precursor, with average dimensions in the range 80-120 μm, having the following general formula (II):

(Ca,K₂,Na₂,Mg)(Al₂O₃)(SiO₂)₁₀.6H₂O (II)

ii) heating the clinoptilolite precursor to a temperature of at least 400° C.; and
iii) subjecting the clinoptilolite precursor obtained from step ii) to a first micronisation in anhydrous conditions to obtain an activated clinoptilolite having average dimensions in the range 1-60 μm.

Surprisingly, the aforesaid method steps allow to activate the zeolite in an unexpected manner compared to the known methods: in fact, as it has been shown, the zeolite dehydrated and activated according to the invention, has a different effect and behaves differently, in biological terms, to zeolites micronised in non-anhydrous conditions.

Moreover, the aforesaid improved behaviour of the zeolite according to the invention is maintained even subsequently to hydration, for example when said zeolite is introduced into a patient's body.

Preferably, following the first micronisation, the average dimensions are between 35 and 58 μm.

According to a preferred embodiment, the aforesaid method further comprises at least one step of subjecting the activated clinoptilolite to a second micronisation, preferably to be conducted in anhydrous conditions, to obtain a clinoptilolite having dimensions in the range 10-35 μm.

As discussed herein before, the at least one micronisation (the first and optionally the second) does not just produce a dimensional reduction of the zeolite particles, but is suitable for increasing the superficial and/or volumetric charge density of the clinoptilolite precursor and/or the activated clinoptilolite depending on the embodiment.

Appropriately, after the zeolite has been micronised (once or twice), it is kept in anhydrous conditions until its use, for example until administered to a patient.

Advantageously, the heating step is conducted at a temperature below 500° C. and, according to a preferred embodiment, the heating step is conducted for a time ranging from 10 minutes to 1 hour, and preferably between 15 and 30 minutes.

The present invention lastly relates to the use of clinoptilolite according to any of the previous variations, and/or obtained using the method according to any of the embodiments illustrated, for the production of a neuro-protective medicine, preferably for treating Alzheimer's and/or Parkinson's disease.

The following examples are for the purposes of illustrating the invention and should in no way be considered as limiting of its relative scope.

### EXAMPLES

### Dehydration of the Clinoptilolite Precursor

In the present invention a clinoptilolite precursor (natural zeolite) was used composed of aluminosilicate of alkaline and alkaline-earth metals having, before treatment, the following general empirical formula (I):

(Ca,K₂,Na₂,Mg)₄Al₈Si₄₀O₉₆.24H₂O (I)

exemplified as a clinoptilolite precursor having the following general formula:

(Ca,K₂,Na₂,Mg)(Al₂O₃)(SiO₂)₁₀.6H₂O (II)

which is treated at 420° C. for 20′ in a rotating drum to obtain its dehydration. Dehydrated clinoptilolite zeolite (ZCD) has in fact been shown to be much more active than the hydrated equivalent in which the interaction of the molecules of water interact with the negative charges present in the channels, with consequent screening effect, reduces its polarity, partially conditioning its capacity to interact with cations, free radicals and toxins. It was then ground to reduce the particles to a maximum dimension of 80-120 μm and its characteristics are as follows:

### Mineralogical Composition

| | |
|---|---|
| Clinoptilolite | 84% |
| Cristobalite | 8% |
| Illite | 4% |
| Plagioclase | 3-4% |
| Quartz | traces |
| Carbonate minerals | traces |

### Physical and Mechanical Data

| Softening temp. | 1260° C. |
|---|---|
| Porosity | 24-32% |
| Melting point | 1340° C. |
| Diameter of pores | 0.4 nm (4 angstrom) |
| Casting point | 1420° C. |
| Relative density | 70% |
| Compression force | 33 MPa |
| Whiteness | 70% |
| Specific weight | 2200-2440 Kg/m³ |
| Mhos hardness | 1.5-2.5 |
| Weight volume | 1600-1800 Kg/m³ |
| Grindability according to VTI kVTI = 1.628 | |
| Appearance and smell | grey-green and odourless |

### Ion Exchange Capacity

| | | |
|---|---|---|
| Total exchanged: | 0.64-0.98 mol/Kg K⁺ | 0.22-0.45 mol/Kg |
| | 0.08-0.19 mol/Kg Na⁺ | 0.01-0.19 mol/Kg |

| | |
|---|---|
| Partial exchange capacity | min. 0.70 mol/Kg |
| Total exchange capacity | 1.2-1.5 mol/Kg |

Steam absorption capacity of the dehydrated rock at

| | |
|---|---|
| relative humidity value of 52% | 7.5-8.5 g H₂O/100 g |
| relative humidity value of 98% | 13.5-14.5 g H₂O/100 g |

### Activation of the ZCD

The ZCD is subjected to a micronisation process based on the collision of the particles with each other causing a contemporary reduction of their size and activation of the same through their structural modification. The size distribution of the particles in the non-activated ZCD shows a trend in the size distribution curve of the particles characterised by a greater breadth and with higher values of D (95) and D (98) in the range 80-115 μm. The ZCD activated once shows a particle size characterised by a narrower distribution and a shift of the D values D (90) and D (98) towards significantly smaller dimensions of 35-58 μm accompanied by an increase in particles of 10-35 μm. The ZCD activated twice shows a further slight reduction of the sizes with D values D (90) and D (98) respectively of 30 and 50 μm and an increase of the particles of 8-30 μm.

The reduction of the size of the particles accompanied by an increase of their active surface was not however suitable for explaining the significant increase in biological activity so a structural study of them was conducted by means of NMR to verify any modifications generated by the micronisation.

### Structure of the Activated ZCD

The powder samples were inserted in rotors of 4 mm in diameter and sealed with Kel-F caps. The NMR silicone spectra were carried out at a frequency of 79.49 MHz with a Bruker Avance 400 spectrometer. The impulse at π/2 was optimised to 3 μs and 400 scans were accumulated with a recycle time optimised to 60 s. For all the samples the rotation speed was set to 12 KHz. The spectra were collected with a number of points in the time domain of 1024 and a Fourier transform applied using 2048 points. Calibration of the chemical shifts was performed using TMS (tetramethylsilane) as an external reference.

The silicone spectra were deconvoluted using a form of Gaussian line and for each peak the intensity, the chemical shift (in ppm) and the width of the line at mid-height (Δν_{1/2}) obtained from the experimental spectra were inserted as initial parameters for deconvolution. By using the spectral deconvolution method the integrals of all the peaks composing the spectrum were obtained.

The analysis was conducted on samples of non-activated ZCD, ZCD activated once and activated twice.

The clinoptilolite zeolite which the present invention relates to shows two centres of symmetry (tetrahedral) which cause overlapping of the resonance bands of the various units. In fact, in the spectra obtained 5 peaks can be observed attributed to the units (resonance bands) Q0 (−112.5 ppm), T (1Al o 0Al) (−106.7 ppm), T (2Al or 1Al) (−100.82 ppm), T (3Al o 2Al) (−96.29 ppm) and shoulder (−87.28 ppm) respectively. The Q0 units are silicone atoms which have as next nearest neighbours (atoms bound to them by an oxygen bridge) 4 atoms of silicone; the T(1Al) units are silicone atoms which have as next nearest neighbours 3 atoms of silicone and 1 atom of aluminium; the T(2Al) units are silicone atoms which have as next nearest neighbours 2 atoms of silicone and 2 atoms of aluminium; the T(3Al) units are silicone atoms which have as next nearest neighbours 1 atom of silicone and 3 atoms of aluminium; lastly, the T(4Al)Q4 units are silicone atoms which have as next nearest neighbours 4 atoms of aluminium.

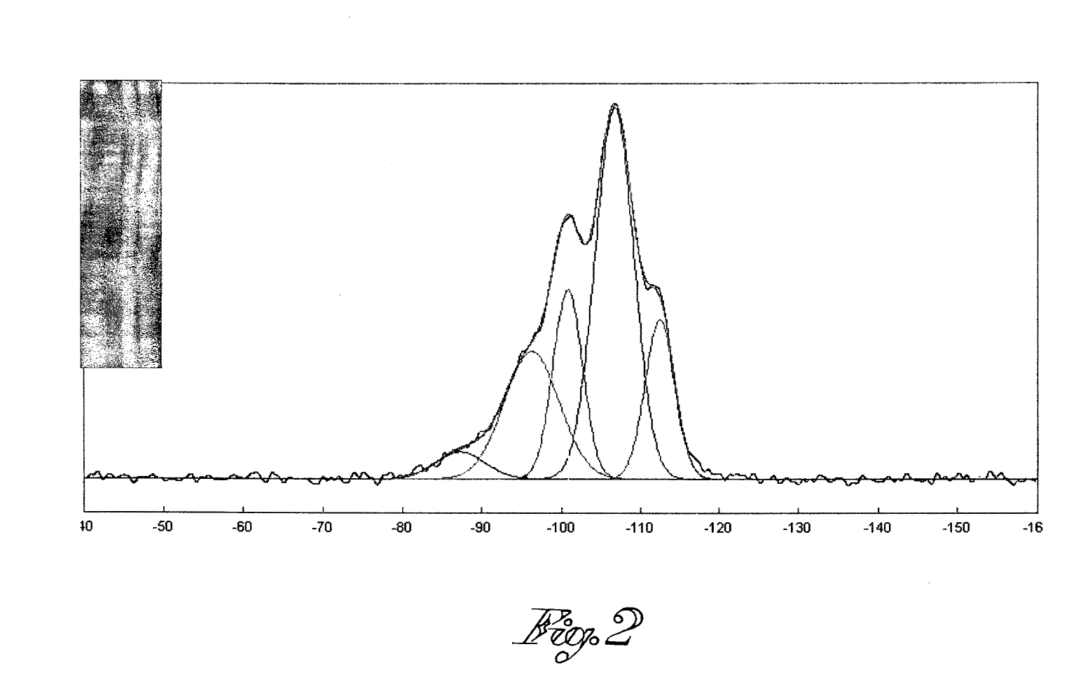

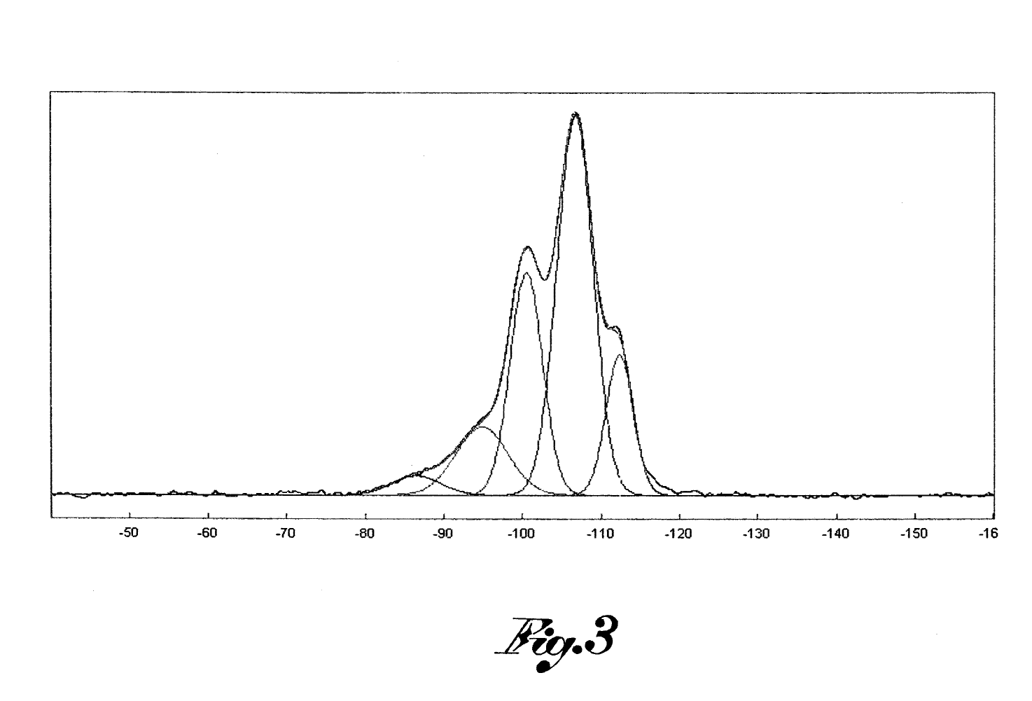

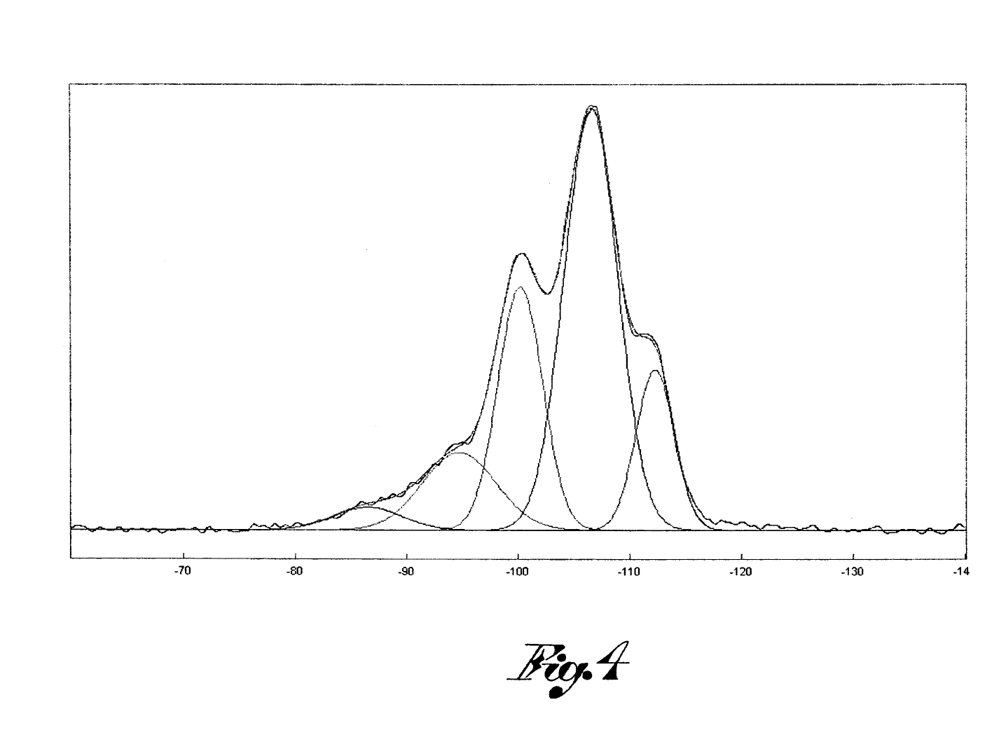

Plotting the spectra one over the other as in FIG. 1 some differences can be seen as regards the intensity of the units Q0, T(2Al or 1Al) and T(3Al or 2Al) while T81Al or 0Al) and the shoulder are quite constant in all samples. Each spectrum was deconvoluted to obtain the areas of the 3 units separately. The deconvoluted spectra together with the individual components were laid over the experimental spectra and are shown in FIG. 2, non-activated zeolite (Sample A), FIG. 3 (zeolite activated once (Sample B) and FIG. 4 zeolite activated twice (Sample C).

Table 1 shows the results of the spectral deconvolution, in other words the peak areas of the units Q0, T (1Al or 0Al), T (2Al or 1Al), T (3Al or 2Al), shoulder (quantitative data).

TABLE 1

| | | T (1Al or | T (2Al or | T (3Al or | |
|---|---|---|---|---|---|
| | Q0 | 0Al) | 1Al) | 2Al) | shoulder |
| Sample | (−112.5 ppm) | (−106.7 ppm) | (−100.82 ppm) | (−96.29 ppm) | (−87.28 ppm) |
| A | 14.42 | 43.65 | 16.76 | 21.18 | 4.0 |
| B | 13.19 | 46.81 | 24.68 | 12.15 | 3.18 |
| C | 13.06 | 48.19 | 23.07 | 12.29 | 3.39 |

Sample A shows values decidedly higher than the unit T (3Al or 2Al) and decidedly lower than T (2Al o 1Al). Such trend shows that the activation to which the ZCD is subjected causes a modification of the structure of the crude zeolite which in sample B subjected to a single activation shows a relatively modest increase of 7.2% of T (1Al or 0Al), a significant increase of T (2Al or 1Al) of 47.7% and a reduction of T (3Al or 2Al) of 42.5% and of the shoulder of 20.0%, while Q0 remains practically constant. In the ZCD activated twice (sample C) a shift of the increase towards T(1Al or 0Al) is shown with a 10% increase compared to sample B, while T(2Al or 1Al) increases by 37.6% and T(3Al or 2Al) decreases by 41.9% and the shoulder by 15.2%. It is evident that the collisions between the particles entail a transformation of T (3Al or 2Al) and of the shoulder in T (1Al or 0Al) but above all in T (2Al or 1Al).

Such trends clearly show that the increase in activity of the activated particles derives from the modification of the structure of the zeolite which brings about a reduction of the number of silicon/aluminium bonds with consequent increase of the atoms of oxygen which support negative charges responsible for the interactions with heavy metals, free radicals and toxins in the channels.

### Particle Size

Measurement of the particle size distribution was carried out with a Malvern Mastersizer 2000, an appliance using a laser diffraction technique (also called LALLS). Unit of dispersion for suspensions Hydro S (volume 50-120 ml). The analysis was carried out in H₂O demineralised with a centrifugal pump and stirrer positioned at 2800-3000 rpm, ultrasound probe in a tank turned on during measurement to improve the disaggregation of the particles. The results obtained are shown in Tables 2 and 3.

TABLE 2

| Sample | D(10) | D(20) | D(30) | D(40) | D(50) | D(60) | D(70) | D(80) | D(90) | D(95) | D(98) |
|---|---|---|---|---|---|---|---|---|---|---|---|
| A | 2.43 μm | 3.71 μm | 5.46 μm | 8.14 μm | 12.30 μm | 18.26 μm | 26.51 μm | 38.79 μm | 61.39 μm | 84.96 μm | 115.20 μm |

TABLE 3

| Sample | D(10) | D(50) | D(90) | D(98) |
|---|---|---|---|---|
| B | 2.43 μm | 10.99 μm | 34.35 μm | 52.33 μm |
| C | 2.11 μm | 8.42 μm | 30.80 μm | 50.53 μm |

Examination of the data clearly shows that the ZCD activated once (Sample B) shows a particle size characterised by a narrower distribution and a shift of the maximum values towards decidedly lower values of 50-60 μm accompanied by an increase in particles of 10-34 μm. The ZCD activated twice (sample C) shows a further slight reduction of the maximum dimensions and an increase of the particles of 8-30 μm.

### Determination of the ζ Potential

Zeta potential was measured using a Malvern Zetasizer Nano, Laser Doppler Electrophoresis method. The powders were dispersed in H₂O (5 mg in 4 ml) demi and analysed with a 0.75 ml capillary cuvette measured using the PALS method (Phase Analysis Light Scattering).

The analytical data shows a negative potential which indicates the presence of negative charges on the particles and an increase, even if slight, in proportion to the extent of activation: Sample A (non-activated) −26.2, Sample B (activated only once) −27.8 and Sample C (activated twice) −28.4. Such trend indicates an increase of the negative charges generated by micronisation and confirms the evidence of the structural study under the NMR.

### Mesoporosity and Surfaces of the Particles

The surface area and porosity of the particles was measured using a Quantachrome NOVA, based on the gas physisorption method. Measurement was performed after 16 hours of degassing in a vacuum at 300° C.; the adsorption and desorption isotherm was measured with nitrogen gas at a temperature of 77K (liquid N₂). The Specific Surface Area (SSA) was calculated with BET multipoint in the field 0.05<P/P0<0.18 in adsorption while Mesoporosity was calculated on the desorption isotherm using both the BJH and DFT methods.

Analysis showed that the clinoptilolite zeolite which the present invention relates to is characterised by a mesoporosity of the dimensions of 38-39 Å and by a surface of 27-28.5 m²/g. The micronisation does not cause a net modification of the mesoporosity while there is a slight increase of the surface showing that the increase of activity is not due mainly to the mesoporosity and to the active surface of the particles but to the modification of the structure of the same with increase of the charges, as shown by the NMR study.

### Transfer Capacity

The transfer capacity of the cations present in the particles which neutralise the negative charges present on the aluminosilicate was measured both so as to verify the possibility of release of potentially toxic ions, as reported by A. Lelas and I. Cepanec in WO 2009/133413 A1, and so as to confirm the increase of the polarisation of the particles after activation.

The measurements were carried out at pH 2 and pH 7 to simulate the conditions of the stomach and human intestine without adding other components of gastric juices which might interfere by forming low solubility salts with many cations, such as phosphates. 0.5 g of sample was suspended in 100 ml of deionised water at pH 7 or in HCl 10⁻² N, stirred constantly for 1 hour and then left to decant and stabilise overnight. The suspension was then centrifuged and the supernatant separated from the solid, filtered and subjected to Atomic Absorption (AA) analysis using a Perkin-Elmer Analist 100.

The transfer is greater at pH 2 for all the cations and in particular for Na⁺, K⁺, Mg²⁺ and Ca²⁺, in relation to the structure, confirming that the silicoaluminate behaves like a strong conjugated base of the corresponding weak aids and can therefore be protonated. The non-activated zeolite (Sample A) shows the greatest transfer capacity, especially of the alkaline-earth metals, while the first activation (Sample B) causes the quantity of alkaline-earth metals transferred to decrease significantly, such values being further decreased after the second activation (Sample C). Such trend indicates a greater electrostatic interaction capacity of the activated zeolites which confirms an increase of the negative charges related to the structural modifications reported above deriving from micronisation.

All the potentially toxic cations, such as the heavy metals, are transferred in concentrations to the order of ppb per kg of zeolite confirming the high degree of purity of the starting material and demonstrating that, at least in the case of the zeolite which the present invention relates to, no purification is needed. The quantities transferred are even lower at pH 7, as may be expected, in that the hydrolysis reaction of the structural silicoaluminates is not so marked. Confirmation is provided by the pH value given to the water by the powder placed in suspension which tends to neutral.

At pH 7 a marked reduction of transfer occurs which shifts towards the alkaline metals for all samples, further confirming that the extent of the activation conditions the transfer; in fact the zeolites activated twice show even lower values of transferred metals.

### Cation Exchange Capacity

0.1 g of sample of zeolite were suspended in 100 ml of deionised H₂O at pH 7 or in solution at pH 2 of HCl containing 1 ml of one of the standard solutions at 1000 mg/l of the metals to be analysed so as to have a final concentration of 10 mg/l of metal. The dispersion was then agitated for about two hours and left to decant overnight. The supernatant was separated and filtered on a 4 μm filter. The analysis was carried out using a Perkin-Elmer Analist 100 atomic absorption spectrophotometer.

The pH significantly influences cation exchange which is higher at pH 7 than at pH 2. At acid pH exchange activity remains high however, demonstrating that even the protonated zeolite is an excellent exchanger. Such trend is particularly evident for lead which bonds at pH 2 for about 75% of the initial quantity while at pH 7 it bonds totally. For mercury too, the exchange capacity is significantly higher to that shown by the highly mesoporous particles of A. Lelas (52 ppb for 1 g of powder) with values 100 times higher around 6 mg for 1 g of activated zeolite. The cation exchange capacity increases markedly with activation and consequently the non-activated sample A shows decidedly lower values to those of sample B activated only once, while sample C is the most active of all.

The activated zeolites which the present invention relates to show a scale of selectivity illustrated below:

Cs⁺>NH₄⁺>Pb²⁺>Ba²⁺>Cu²⁺>Hg²⁺>Cd²⁺>Ni²⁺>Co²⁺>K⁺>Na⁺>Ca²⁺>Mg²⁺>Zn²⁺ highlighting a very low interaction with essential oligo-elements from a biological point of view such as zinc.

### Inhibition of ROS Production by the Zeolite Clinoptilolite.

The effect of the zeolite activated once (Sample B) and twice (Sample C) was studied in an experimental mitochondrial ROS production model using a confocal microscope in live neuronal cells as described below. This model permitted measurement of the levels of ROS generated in the mitochondria.

In brief, the neuroblastoma cellular line SH-SY5Y was initially differentiated into cells with neuronal phenotype by in vitro treatment with retinoic acid for 7 days (Uberti et al. 1997); subsequently, using the method described by Koopman et al. (Cytometry A 2006 69(12):1184-92)5-chloromethyl-2′,7′-dichlorodihydrofluorescein (CM-H₂DCF) was added to the culture medium and its oxidative conversion in CM-DCF within the cells was monitored by measuring the luminous intensity with a confocal microscope and a real time image. The MITOTRACKER DEEP RED was used to localise the mitochondria in the cells. The cells were subsequently excited with increasing laser intensity and the intensity of the CM-DCF fluorescence was measured in the affected areas (mitochondria). The intensity of emission of the fluorescence was calculated as the value of the average level of green per pixel and corrected for the background.

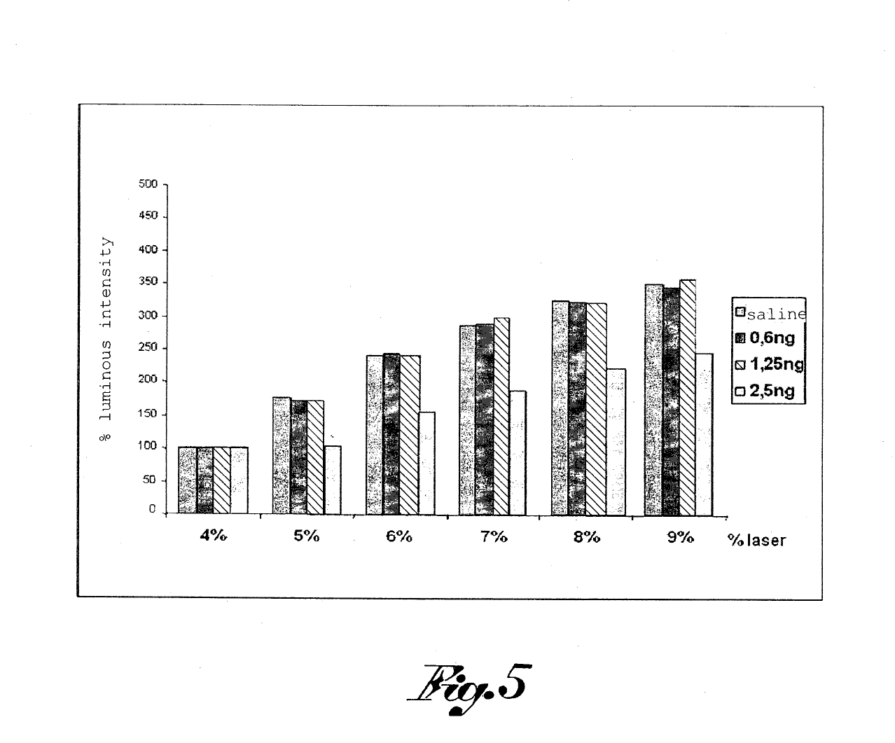

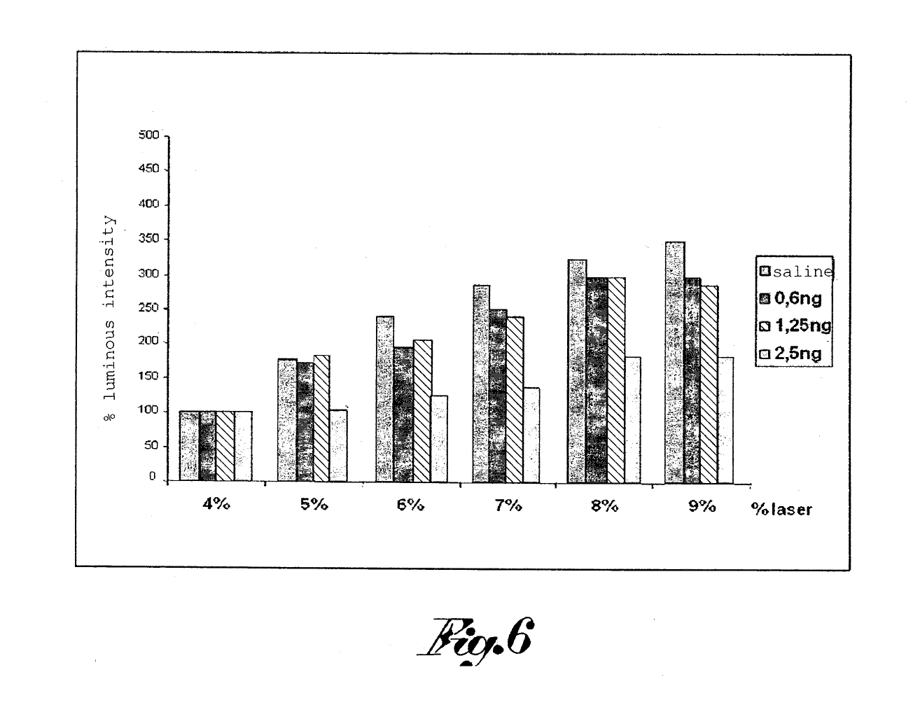

In our study the cells were pre-incubated with increasing concentrations both of Sample B and Sample C (from 0.6 ng/ml to 2.5 ng/ml) for 24 or 48 hours before excitation of the laser. We found that at the concentration of 2.5 ng/ml both Sample B and Sample C prevented induction by the laser of the production of ROS in the mitochondria of differentiated SH-SY5Y neuronal cells. FIGS. 5 and 6 show the results of the experiments. For Sample C the effect was already evident after exposure of 24 hours.

### Neuro-Protective Effects of Zeolite in an Experimental Model of Cell Death Induced by ROS

The cellular line SH-SY5Y of neuroblastoma was differentiated with retinoic acid for a week so as to acquire the neuronal phenotype. After differentiation into neuronal-type cells, the cells were exposed to increasing concentrations of non-activated zeolite, sample A, zeolite activated once, sample B, and twice, sample C (0.5-5.0 ng/ml) for 24 hours. Cell vitality was monitored after brief exposure of the cells to H₂O₂.

Previous experiments conducted in our laboratory (Uberti e al. 2002) showed that exposure of these cells to increasing concentrations of H₂O₂ in a range from 0.5 mM to 2 mM, for varying periods of time from 1 min to 20 minutes led to a significant increase in the production of ROS. The effect proved to be concentration and time-dependent, and cell death was more likely to be identified 24 hours after exposure to the H₂O₂. The experimental model of neuronal death induced by ROS used in our studies has been recognised and approved by the scientific community for many years. On the basis of these assumptions, the cells were exposed to H₂O₂ for 5 minutes and then fresh medium was added to the cells for another 24 hours. The H₂O₂ caused a reduction of cell life of about 70% compared to the non-treated cells considered as a control.

As shown in FIG. 6, we find that treatment with increasing concentrations of Sample B and Sample C prevents the dose dependent cell death induced by H₂O₂.

The compounds tested show differing efficacy with the zeolite activated twice proving more active than the zeolite activated once.

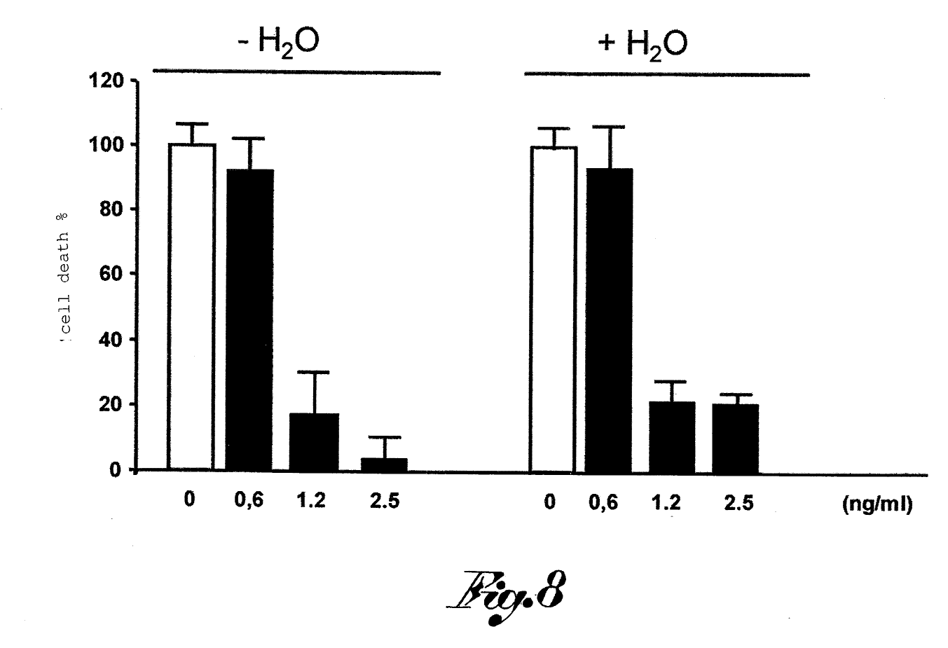

The non-activated zeolite did not how any neuro-protective effects. FIG. 8 shows that the dehydration of the zeolite increases the neuro-protective effect of the compound.

### Methods

### Cell Culture

The neuroblastoma human cell line SH-SY5Y is grown in 1:1 Ham's F12:Dulbecco modified Eagle's medium (DMEM) supplemented with 10% (v/v) of foetal calf serum, 2 mM of glutamine, 50 μg/ml of penicillin and 100 μg/ml of streptomycin and kept at 37° C. in an atmosphere humidified with 5% of CO₂. To differentiate, the cell culture was treated with 10 μM of retinoic acid for 1 week. Growth and differentiation were carried out as per protocol Uberti et al. 1007.

### Pharmacological Treatment

Zeolite was added to the growth medium of the cells 24 or 48 hours before the pulse with H₂O₂, or stimulation with laser light as indicated.

### Assessment of Cell Vitality

Cell vitality was measured using a quantitative colorimetric method with MTT (3-(4,5-dimethylthiazol-2,5-diphenyltetrazolium bromide), which shows the mitochondrial activity of live cells. The differentiated neuronal cells SH-SY5Y in the 96-well plate are kept for 5 min. with H₂O₂ and subsequently MTT 500 μg/ml (final concentration) is added to each well, the cells are then incubated for another 3 hours at 37° C., MTT is removed and the cells lysed with dimethyl sulphoxide. Absorbance at 595 nm was measured using a microplate reader Bio-Rad 3350. The control cells were treated in the same way but without the H₂O₂ and the differences in absorbance values were expressed as control percentage.

### Detection of the Reactive Oxygen Species (ROS)

The cellular line SH-SY5Y of neuroblastoma was first differentiated in a with neuronal-type phenotype by treatment with retinoic acid for 7 days according to Koopman et al. 2006, 5-chloromethyl-2′,7′-dichlorodihydrofluorescein (CM-H₂DCF) was added to the cells and its oxidative conversion in CM-DCF was monitored by means of a light intensity function with the confocal microscope and a real time image after increasing the intensity of the laser. The MITOTRACKER DEEP RED was added to the cells to localise the mitochondria. The cells were subsequently excited with increasing laser intensity and the intensity of the CM-DCF fluorescence was measured selectively in the mitochondria.

The intensity of emission of the fluorescence was calculated as the value of the average level of green per pixel and corrected for the background.

### CONCLUSIONS

The results reported in the experimental part reveal an unexpected property of the zeolite according to the present invention as anti-oxidant in the mitochondria and a marked neuro-protective effect suggesting that the zeolites obtained with the treatment herein described have a therapeutic action in those neuropathologies in which the production of ROS plays a relevant role.

The neuro-protective effect is dependent on the activation of the compound and in fact for Sample C (activated twice) is greater than for Sample B (activated once). In our model, the non-activated Zeolite does not display neuro-protective properties. The removal of water by means of heat increases in an evident manner the neuro-protective activity induced by the zeolite of the present invention as is evident from the experimental data relative to Sample B and to the corresponding hydrated sample activated only once such as B.

Even if not previously specified, a person skilled in the art may, by resorting to the expertise typical of the sector, vary or replace some of the aspects indicated above with other technically equivalent elements.

These variations or replacements also fall within the scope of the invention as defined by the following claims.

Moreover, any alternative illustrated in relation to a particular embodiment may be realised independently of the other variations described.

1. A dehydrated and micronised clinoptilolite, characterised by a negative ζ potential equal to or smaller than −26.6 mV.

2. The clinoptilolite of claim 1, wherein such potential is comprised between −26.6 mV and −30 mV.

3. The clinoptilolite of claim 1, having average dimensions in the range 1-60 μm.

4. A method for the preparation of an activated clinoptilolite comprising, in succession, the steps of:
i) providing a clinoptilolite precursor, with average dimensions in the range 80-120 μm, having the following general formula (II):
(Ca,K₂,Na₂,Mg)(Al₂O₃)(SiO₂)₁₀.6H₂O (II)
ii) heating the clinoptilolite precursor to a temperature of at least 400° C.; and
iii) subjecting the clinoptilolite precursor of step ii) to a first micronisation in anhydrous conditions to obtain an activated clinoptilolite having average dimensions in the range 35-58 μm; and
iv) subjecting the activated clinoptilolite to a second micronisation to obtain a clinoptilolite having dimensions in the range 10-35 μm;
micronisation being performed by colliding clinoptilolite particles with each other

5-6. (canceled)

7. The method of claim 4, wherein the second micronisation is performed under anhydrous conditions.

8. The method of claim 4, further comprising a step of maintaining the clinoptilolite in anhydrous conditions until its use.

9. The method of claim 4, wherein the heating step is conducted at a temperature between 420° C. and 450° C.

10. The method of claim 4, wherein the heating step is conducted for a time ranging from 10 minutes to 1 hour.

11. (canceled)

12. A clinoptilolite obtained using the method of claim 4.

13. A composition comprising a pharmacologically acceptable carrier and the clinoptilolite of claim 1.

14. pathologies A method of treating a neurological pathology in a subject, comprising administering to the subject the clinoptilolite of claim 1.

15. The method of claim 15, wherein the neurological pathology is Alzheimer's or Parkinson's disease.
